(19) United States
(12) Patent Application Publication
(10) Pub. No.: US 2015/0374485 A1
(43) Pub. Date: Dec. 31, 2015

(54) TARGETED PERFORATIONS IN ENDOVASCULAR DEVICE

(71) Applicant: Cordis Corporation, Fremont, CA (US)

(72) Inventor: Ramesh Marrey, Pleasanton, CA (US)

(73) Assignee: Cordis Corporation, Fremont, CA (US)

(21) Appl. No.: 14/317,597

(22) Filed: Jun. 27, 2014

(51) Int. Cl. A61F 2/07; A61L 27/06; A61F 2/89

(52) U.S. Cl. CPC A61F 2/07; A61F 2/89; A61L 27/06; A61F 2002/072; A61F 2210/0004; A61F 2210/0014; A61L 2400/16

(57) ABSTRACT

Various embodiments for an endovascular device (and variations thereof) that prevents focalized edge (or end) restenosis. In particular, these improvements would mitigate or prevent focalized restenosis at the ends of the device. The designed-in restenotic regions would be circumferentially and axially distributed so that graft patency is not compromised.

### BACKGROUND

It is well known to employ various intravascular endoprostheses delivered percutaneously for the treatment of diseases of various body vessels. These types of endoprosthesis are commonly referred to as stents. A stent is a generally formed longitudinal tubular device of biocompatible material, such as stainless steel, cobalt-chromium, nitinol or biodegradable materials, having holes or slots cut therein so they can be radially expanded, by a balloon catheter or the like, or alternately self-expanded within the vessel. Stents are useful in the treatment of stenosis, strictures or aneurysms in body vessels such as blood vessels. These devices are implanted within the vessel to reinforce collapsing, partially occluded, weakened or abnormally dilated sections of a vessel. Stents are typically employed after angioplasty of a blood vessel to prevent restenosis of the diseased vessel. While stents are most notably used in blood vessels, stents may also be implanted in other body vessels such as the urogenital tract and bile duct.

Stents generally include an open flexible configuration. This configuration allows the stent to be inserted through curved vessels. Furthermore, the stent configuration allows the stent to be configured in a radially compressed state for intraluminal catheter implantation. Once properly positioned adjacent the damaged vessel, the stent is radially expanded so as to support and reinforce the vessel. Radial expansion of the stent can be accomplished by inflation of a balloon attached to the catheter, or alternatively using self-expanding materials such as nitinol within the stent. Examples of various stent constructions are shown in U.S. Pat. No. 4,733,665 filed by Palmaz on Nov. 7, 1985, which is hereby incorporated herein by reference.

Recently, there has been a desire to place a covering of biocompatible material over expandable stents. The covering for the stent can provide many benefits. For example, the covered stent could act as a stent-graft. Intraluminal vascular stent-grafts can be used to repair aneurysmal vessels, particularly aortic arteries, by inserting an intraluminal vascular graft within the aneurysmal vessel so that the prosthetic withstands the blood pressure forces responsible for creating the aneurysm.

### SUMMARY OF THE DISCLOSURE

Applicant notes that there are at least two down-sides to usage of the graft or a combined stent-graft in the vasculature: (a) the graft is believed to occlude side-branches across the length of the treated vasculature and (b) the graft leads to focal edge restenosis, i.e., focalized restenosis at proximal and distal ends of the graft. In fact, edge restenosis is the primary cause of failure of stent grafts. For instance, 87% of stent graft failures in the VIBRANT trial (from the August 2012 publication of “Endovascular Today”) were via focalized edge restenosis. In contrast, 93% of the failures in bare nitinol stents (BNS) exhibited diffused restenosis. The VIBRANT trial is a multicenter, randomized study of the prior GORE® VIABAHN® Device (without heparin, contoured proximal edge; 5-mm device sizes available) versus BNS (multiple brands) in 148 patients (Rutherford classes 1-5), with a primary endpoint of primary patency at 3 years. The mean lesion lengths were 19 and 18 cm, 40% were CTOs, and 62.5% of lesions demonstrated moderate to severe calcification (primarily TASC C and D lesions). Both groups had disappointing primary patency rates of 53% and 58%, respectively, but there were important differences in the patterns of restenosis: 93% of failed BNS had diffuse ISR versus focal edge restenosis in 87% of the failed GORE® VIABAHN® Devices.

Therefore, applicant has recognized that certain improvements can be made to a prosthetic such as a stent-graft to achieve restenotic response at targeted regions. In short, the present invention is an endovascular prosthetic in the form of a graft or stent-graft (and variations thereof) that prevents focalized edge (or end) restenosis. In particular, these improvements would mitigate or prevent focalized restenosis at graft ends. The designed-in restenotic regions would be circumferentially and axially distributed so that graft patency is not compromised.

One embodiment of the present invention may include: an expandable frame having a plurality of hoops disposed about a longitudinal axis extending through the plurality of hoops from a first frame end to a second frame end; a generally cylindrical graft material disposed generally coaxial to the expandable frame about the longitudinal axis from a first graft end to a second graft end, the graft material being connected to the frame at a plurality of locations; and wherein the graft material is configured to include perforations formed on the graft material so that the perforations proximate the first and second graft ends are equal or larger than the perforations that are disposed away from the first or second graft end. The expandable frame may be enclosed by graft material on its outside surface, inside surface or both surfaces.

In the embodiment noted above, the perforations proximate the first and second graft ends generally define respective hoops of perforations proximate the first and second graft ends. Alternatively, the perforations of the graft material define a helical path from the first graft end to the second graft end and a width of such helical path is progressively smaller as the helical path moves away from the first graft end or the second graft end. Further, the expandable frame is disposed on an inner surface of the graft material that is facing the longitudinal axis. Alternatively, the expandable frame can be disposed on the outside surface of the graft; the expandable frame can be sandwiched between two graft materials; or two expandable frames can sandwich graft material.

In such embodiment, the perforations proximate the first graft end or the second graft end comprise a plurality of perforations wherein each perforation defines an opening having an open area AP that has a first aspect ratio range from about 0.1 through about 0.5 of AO1 or AO2, where AO1 or AO2 is end section area one of the first and second graft end perpendicular to the longitudinal axis. Another range for the first aspect ratio could be from about 0.2 to about 0.4. As used herein, AO1 denotes the surface area orthogonal to the longitudinal axis L-L of the first opening of the endovascular prosthetic and AO2 denotes the surface area of the second opening, in which AO1˜AO2 or AO1≠AO2. Alternatively, each of the perforations disposed away from one of the first and second graft ends defines an open area that is progressively smaller from about 0.4 to about 0.9 and could be from about 0.5 to about 0.8 than the open area of the perforations proximate one of the first and second graft ends to define the second aspect ratio range. For example, the ratio of the area AP3/AP2 can be from about 0.4 to about 0.9 and likewise, the ratio of AP4/AP3 is from about 0.4 to about 0.9. The perforations can be of any suitable configuration including but not limited to circular, elliptical, dog-boned or alternate patterns, as long as such configuration complies with the first and second aspect ratios described herein.

In yet another embodiment, perforations may include at least a slit through the graft material and extending generally parallel to the longitudinal axis. It is noted that the at least one slit may be two slits disposed diametrically with respect to the longitudinal axis and spaced apart longitudinally or more than two slits disposed diametrically and staggered longitudinally. Width in circumferential direction of longitudinal slits can be from about 0.1 to about 0.5 times the proximal or distal graft diameter.

The expandable frame may be one of a self-expanding frame or a balloon expandable frame which frame can be of at least a bioresorbable material. The frame may include a series of hoops connected to each other via connectors of the same material as the frame. Alternatively, the frame may include a series of hoops independent from each other so that the hoops are connected indirectly through the graft material. The graft materials may be composed of various polymeric formulations including PET (polyester), Fluoro-polymers such as PTFE and FEP, spun PTFE, and HDPE.

In the case of a graft where no internal or external frame is needed, a thin-film graft made from nitinol can be utilized with either or both of the aspect ratios noted earlier. The “thin-film” material for the graft can be made from well-known chemical deposition or physical deposition techniques. Chemical deposition can be by plating, chemical solution deposition, spin coating, chemical vapor deposition, plasma enhanced vapor deposition, or atomic layer deposition. Physical deposition for thin film manufacturing can be by thermal evaporator, laser deposition, cathodic arc deposition, sputtering, vapor deposition, ion-beam assisted evaporative deposition or electrospray deposition.

These and other embodiments, features and advantages will become apparent to those skilled in the art when taken with reference to the following more detailed description of the exemplary embodiments of the invention in conjunction with the accompanying drawings that are first briefly described.

### BRIEF DESCRIPTION OF DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements), in which:

FIG. 1 illustrates a perspective cut-away view of a stent graft according to one embodiment of the invention.

FIG. 2 illustrates yet another perspective cut-away view of a stent graft according to a second embodiment of the invention.

FIG. 3 illustrates a perspective cut-away view of yet a third embodiment.

FIG. 4A illustrates a perspective cut-away view of yet a fourth embodiment.

FIG. 4B illustrates a cross-sectional view taken along a plane orthogonal to the longitudinal axis L-L.

FIG. 5 illustrates a perspective view of one technique to form the perforations in a split type punch and die mold form.

FIG. 6A illustrates an endovascular prosthetic made using a suitable thin-film material in accordance with the principles of the present invention.

FIG. 6B illustrates a cross-sectional view taken along a plane orthogonal to the longitudinal axis.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human use, although use of the subject invention in a human patient represents a preferred embodiment.

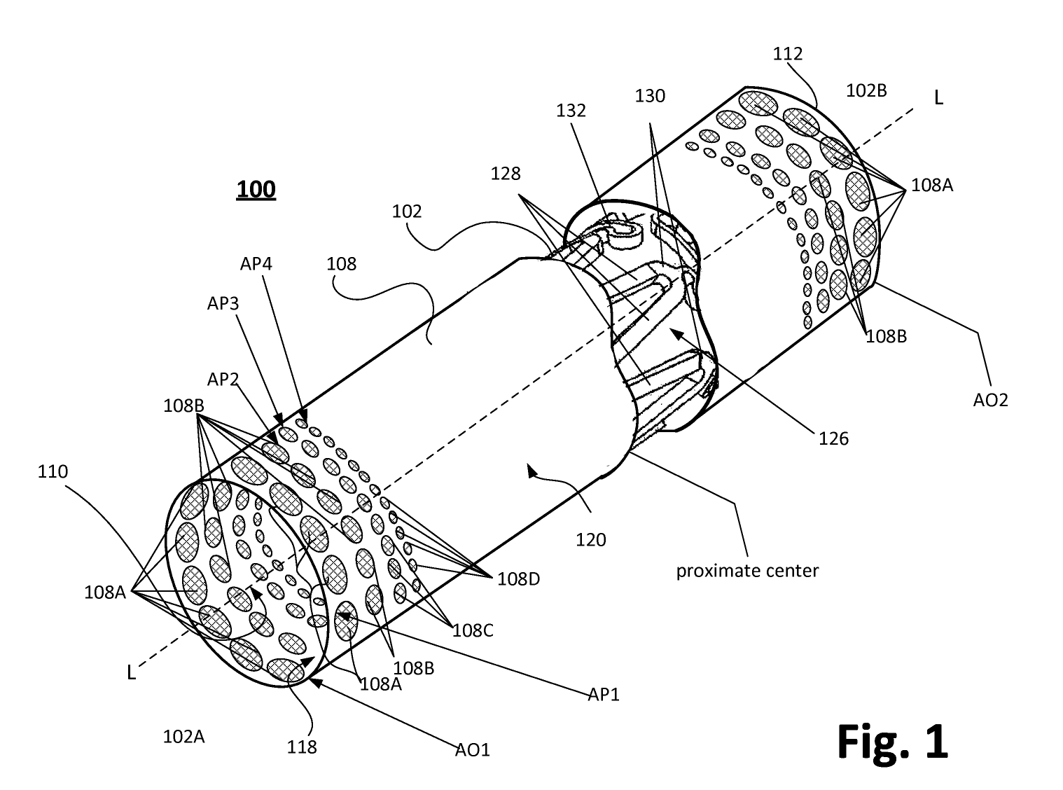

Referring now to the figures wherein like numerals indicate the same element throughout the views, there is shown in FIG. 1 an endovascular prosthetic 100 made in accordance with the present invention. Prosthetic 100 is designed for insertion into a target site within a vessel of a patient, to treat various vascular diseases. The prosthetic 100 has a crimped state (not shown for brevity) for delivery to the target site, and an expanded state, shown in FIG. 1 for implantation within the vessel. Individual parts of the endovascular prosthetic 100 in the form of a “stent-graft” will be described in details, however, a brief description of the overall device would be helpful in understanding the design. As used herein, stent-graft is intended to cover an endovascular device without any supporting frame or an endovascular device with a supporting frame attached to the device.

As shown in FIG. 1, one embodiment, referenced here as 100, of the prosthetic invention is shown. In this embodiment, the prosthetic 100 includes an expandable frame 102 having a plurality of hoops (only one hoop 126 is shown in a partial view) disposed about a longitudinal axis L-L extending through the plurality of hoops from a first end 102A to a second end 102B. The endovascular prosthetic 100 includes generally cylindrical graft material 108 disposed generally coaxial to the expandable frame 102 about the longitudinal axis from a first graft end 110 to a second graft end 112. The graft material 108 can be connected to the frame at a plurality of locations with respect to the expandable frame 102. In particular, the graft material 108 is configured to include perforations 108A, 108B, 108C, 108D and so on formed on the graft material so that the perforations (108A, 108B, 108C, 108D and so on) proximate the first and second graft ends 102A and 102B are larger than the perforations that are disposed away from the first or second graft end.

The embodiment of FIG. 1 shows one perforation pattern where the proximal and distal graft ends include perforations of gradually decreasing size (in the open area of the perforation) or density (of the number of perforation per surface area) along the graft axis L-L. These progressively decreasing perforations in size or density are intended to shift the focalized restenosis at graft ends to a diffused response at the graft ends. These perforations may be present along the entire circumference or for a section of the graft circumference. The perforations can be of different topologies, such as, for example, circular, elliptical or other shapes. In this embodiment, the perforations 108A can be configured such that a first aspect ratio of the area (e.g., AP1) defined by each perforation 108A is about 0.1 to about 0.5 of the area “A” defined by the one of the first or second openings (e.g., AO1 or AO2) of the endovascular prosthetic 100. Additionally, perforations (e.g., 108B, 108C, and 108D) disposed away from one of the first and second graft ends defines an open area (i.e., an area not covered by graft material) that is smaller by about 0.4 to about 0.9 than its longitudinally adjacent perforation. This progressive decrease in area is the second aspect ratio for the perforations, and this ratio can apply to for instance AP2/AP1 or AP3/AP2 or AP4/AP3 as shown in FIG. 1. For example, the perforation 108B may have a decrease in its area governed from the second aspect ratio. The decrease in the area from 108A to 108B to 108C can be progressive or in a predetermined decrement level governed by the second aspect ratio. In particular, as shown in FIG. 1, perforations of similar sizes are arranged in a hoop like pattern such as shown here in for perforations 108A disposed about the longitudinal axis L-L. Moving away from the first opening 102A or second opening 102B, the perforations are configured such that they are smaller in size (e.g., in the opening area or area of a hole of the perforation). For example, perforations 108B are smaller in the area AP2 (defined by the opening of each perforation) as compared to the area AP1 for each of perforations 108A which are closer to the first graft opening 102A (or second graft opening 102B). Similarly, perforations 108C and 108D are smaller (with respective area AP3 and AP4) with reference to perforations 108A and 108B.

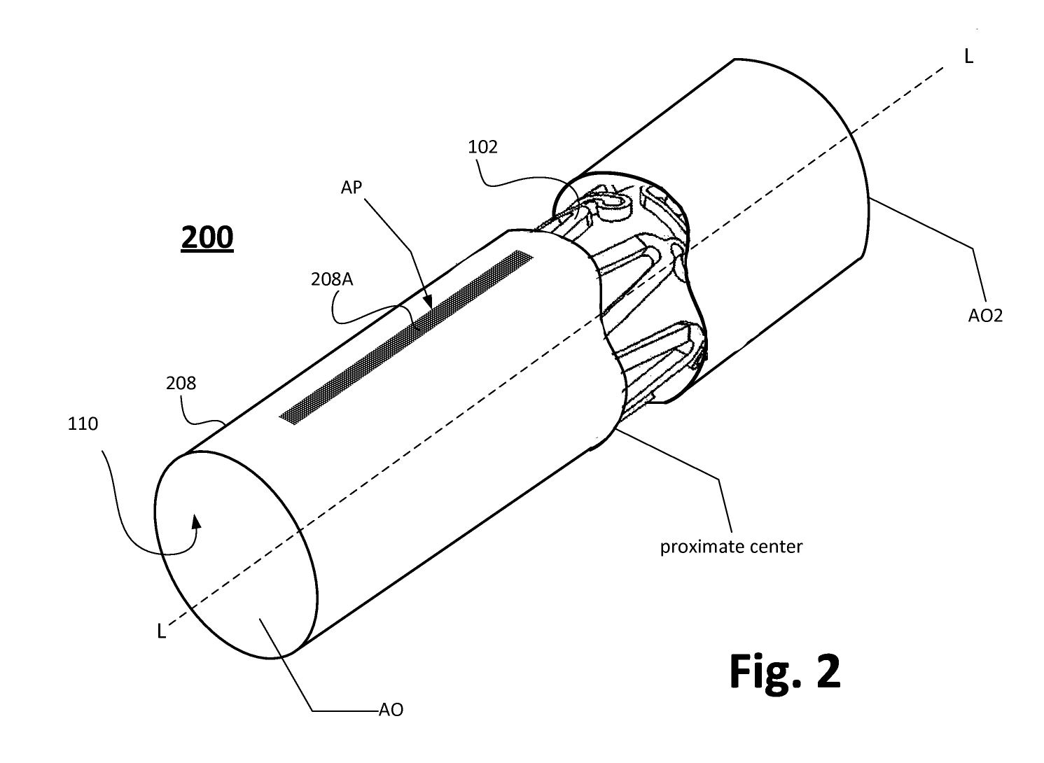

FIGS. 2-4 show perforation patterns intended to distribute the restenotic response along a predominant length of the graft 208, 308, or 408. FIG. 2 shows a straight perforation 208A running along a substantial length of the graft 208. By designing a single perforation 208A, this configuration allows for a single region of restenotic response along any cross-section across graft length. Therefore, the remainder of the graft cross-section would be able to maintain patency.

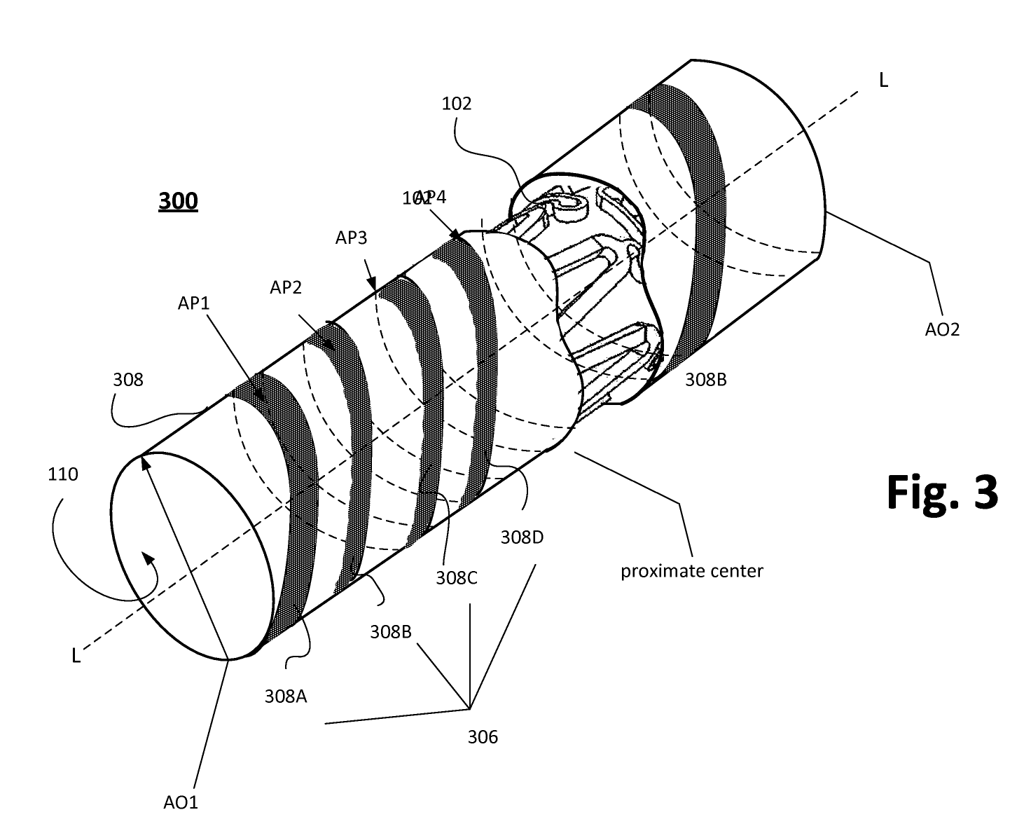

Alternatively, FIG. 3 shows a continuous helical perforation formed by helical segments 308A-308D, which can be viewed as a single helix or a series of discontinuous helical segments—this pattern allows a single region of restenotic response per cross-section along graft length. The continuous or discontinuous helix may include segments which decrease in width to provide for differing open area AP1, AP2, AP3, AP4 and so on. In one embodiment, the magnitude of AP1 is greater than AP2 which is greater than AP3 which is greater than AP4.

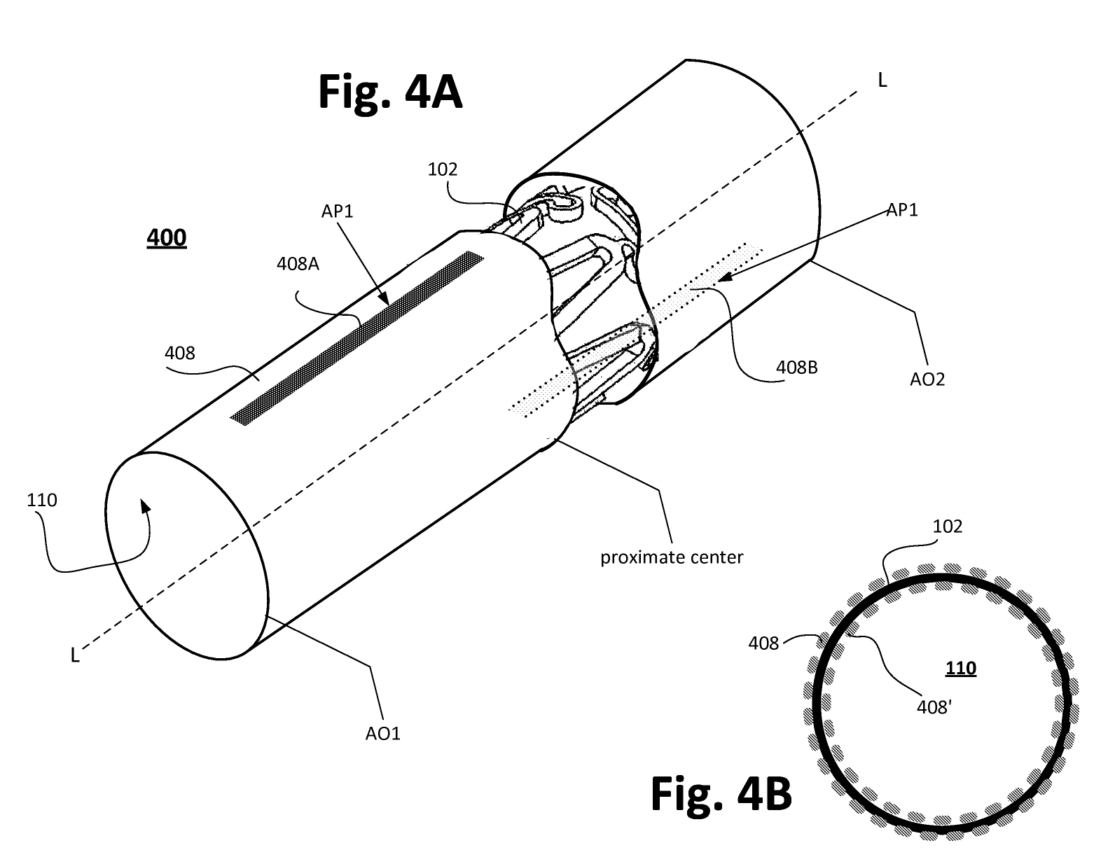

While FIG. 2 shows a single perforation 208A, an alternative graft embodiment 408 in FIG. 4 may include multiple perforations 408A dispersed circumferentially and axially across the graft 408. As an example, FIG. 4 shows two straight perforations 408A disposed diametrically opposed to each other with respect to the longitudinal axis L-L. This alternative can be similarly extended to more than two perforations with appropriate circumferential phase shift. Instead of two straight perforations, the perforations can be in the form of a curve or curvilinear. The same principle can be applied to the helical pattern, where the pattern could be discontinuous, segmented, or in the form of contra-rotating helical perforations. As with the other embodiments, the perforation 408A may be in the form an elongated open area such as for example, a rectangle or a polygon including a four-sided polygon with two converging sides.

In each of the embodiments described herein, frame 102 may be a self-expanding expandable stent or a balloon expandable stent. The frame 102 is a tubular member having a first end 102A and a second end 102B. The frame 102 has an interior surface 110, which is not pointed out in FIG. 1 because it is obstructed, and an exterior surface 112. Frame 102 can be made from an elastic material. Prosthetic 100 further includes a tubular flexible porous graft material 108, preferably expanded PTFE, extending along the interior of the outer stent. Graft material 108 has a first end 110, a second end 112, an interior surface 118 and an exterior surface 120. In one embodiment, the front and back ends of the graft member could be folded over and bonded to the front and back ends of the the expandable frame to form cuffs at respective ends of the frame.

Frame 102 is preferably made from a suitable biocompatible material such as balloon expandable metal alloy or a superelastic alloy such as Nitinol. Most preferably, frame 102 is made from an alloy comprising from about 50.5% (as used herein these percentages refer to atomic percentages) Ni to about 60% Ni, and most preferably about 55% Ni, with the remainder of the alloy Ti. Preferably, the stent is such that it is superelastic at body temperature. The superelastic design of the expandable frame makes it crush recoverable which, as discussed above, is useful in treating many vascular problems.

Referring back to FIG. 1, this figure illustrates the prosthetic 100 in its partially expanded state with frame 102 which includes struts, loops and bridges. Frame 102 is a tubular member having front and back open ends 102A and 102B and a longitudinal axis L-L extending therebetween. The tubular member has a crimped diameter (not shown for brevity) and a second larger expanded diameter, (not shown for brevity) as compared to an intermediate diameter (FIG. 1). As seen from FIG. 1, the hoops 126 include a plurality of longitudinal struts 128 and a plurality of loops 130 connecting adjacent struts, wherein adjacent struts are connected at opposite ends via hoop bridges 132 so as to form an S shape pattern.

The stents can be cut from a tube or wound from a wire on a mandrel. Thereafter, the stents can be expanded in the duct or vessel of a host by a separate mechanism (e.g., balloon) or by utilization of a material that self-expands upon predetermined implantation conditions. The stent can be formed from a suitable biocompatible material such as, for example, polymer metals and other biocompatible materials which may be bioabsorble. Preferably, stents are laser cut from small diameter tubing from biocompatible metals such as shape memory materials or balloon expandable materials. Details of this particular embodiment of the stent can be gleaned from U.S. Pat. No. 8,328,864, which is hereby incorporated by reference herein.

Although the stent frame has been shown and described as being connected via bridges, one embodiment of the stent frame includes a plurality of discrete hoops that are not connected directly to other stent hoops via stent bridges but indirectly by virtue of each hoop being attached to the graft material (e.g., sutured, glued or retained between inner and outer graft materials).

Graft material 108 of prosthetic 100 is preferably made from a suitable material such as, for example, PTFE, ePTFE, Dacron, PET (polyester), Fluoro-polymers such as PTFE and FEP, spun PTFE, HDPE, and combinations thereof. Either or both of the graft and stent can be formed from biodegradable polymers such as polylactic acid (i.e., PLA), polyglycolic acid (i.e., PGA), polydioxanone (i.e., PDS), polyhydroxybutyrate (i.e., PHB), polyhydroxyvalerate (i.e., PHV), and copolymers or a combination of PHB and PHV (available commercially as Biopol®), polycaprolactone (available as Capronor®), polyanhydrides (aliphatic polyanhydrides in the back bone or side chains or aromatic polyanhydrides with benzene in the side chain), polyorthoesters, polyaminoacids (e.g., poly-L-lysine, polyglutamic acid), pseudo-polyaminoacids (e.g., with back bone of polyaminoacids altered), polycyanocrylates, or polyphosphazenes. As used herein, the term “bio-resorbable” includes a suitable biocompatible material, mixture of materials or partial components of materials being degraded into other generally non-toxic materials by an agent present in biological tissue (i.e., being bio-degradable via a suitable mechanism, such as, for example, hydrolysis) or being removed by cellular activity (i.e., bioresorption, bioabsorption, or bioresorbable), by bulk or surface degradation (i.e., bioerosion such as, for example, by utilizing a water insoluble polymer that is soluble in water upon contact with biological tissue or fluid), or a combination of one or more of the bio-degradable, bio-erodable, or bio-resorbable material noted above.

In certain applications where a fabric or a polymeric material is not desired, the graft material 108, 208, 308 or 408 can be formed by a suitable thin-film deposition technique over a substrate such as an expandable frame (self-expanding or balloon expandable stent). In this configuration with the thin-film, the expandable frame can be disposed on the outside surface of the thin-film (acting as a graft); the expandable frame can be sandwiched between two thin-film graft materials (FIG. 4B with outer graft 408 and inner graft 408′); or two expandable frames can sandwich the thin-film graft material.

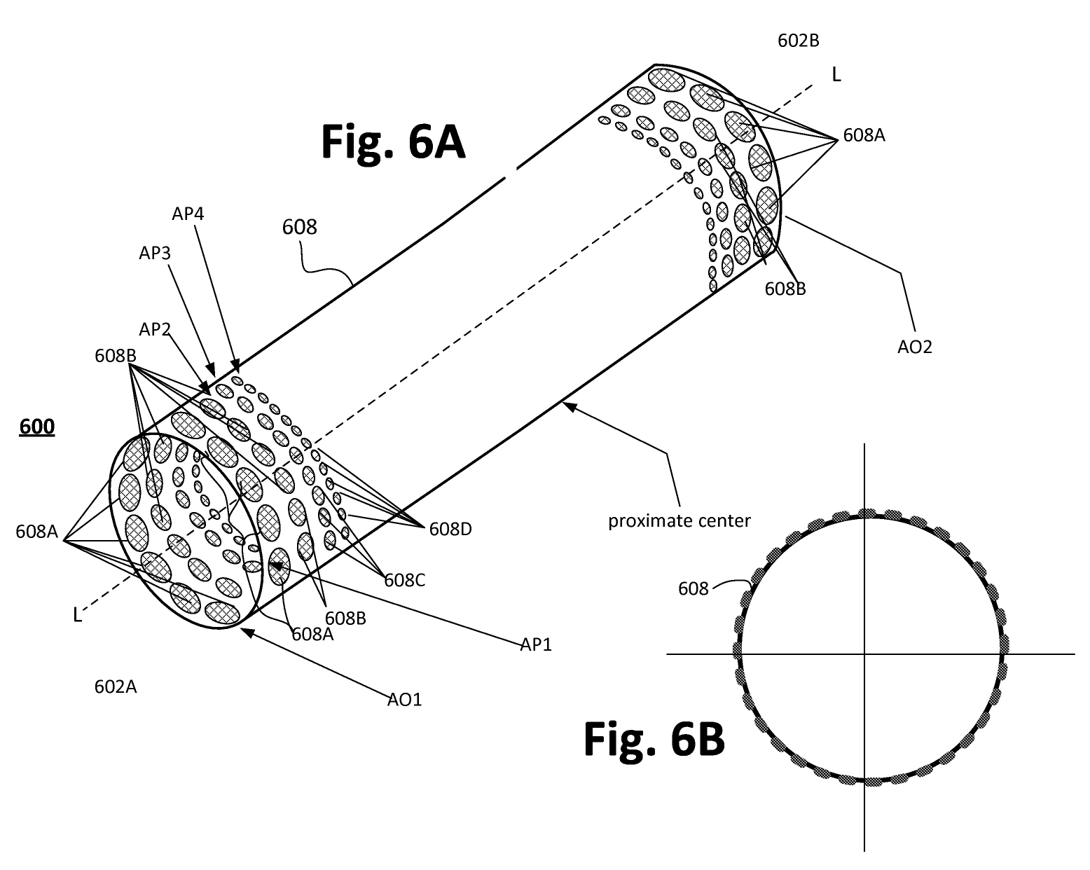

Alternatively, in applications that may require a very thin graft in the pre-deployment profile, the stent as a substrate is eliminated completely from the prosthetic thereby resulting in a prosthetic formed from a thin-film of materials such as biocompatible metals or pseudometals (FIGS. 6A and 6B).

FIG. 6A illustrates an embodiment of such thin-film prosthetic 600 formed from a thin-film expandable graft 608 that does not require a graft material. The endovascular prosthetic 600 has first end 602A, second end 602A, perforations 608A, 608B, 608C, 608D and so on towards the proximate center of the prosthetic 600. It is noted that the nomenclatures for AO1, AO2, AP1, AP2, AP3, AP4 and so on have the same meanings noted in FIGS. 1-4. In such frameless graft configuration, the thin-film graft 608 itself would be imbued with both characteristics of the stent and graft combination yet with only a single unitary component in the form of a thin-film graft 608 (FIG. 4B). In the preferred embodiment, the thin-film graft may have a thickness from about 0.1 micron to about 25 microns of a suitable material. The thin-film graft 608 can be formed as a single layer or multiple layers using well-known chemical deposition or physical deposition techniques. Briefly, chemical deposition can be by plating, chemical solution deposition, spin coating, chemical vapor deposition, plasma enhanced vapor deposition, or atomic layer deposition. Physical deposition for thin film manufacturing can be by thermal evaporator, laser deposition, cathodic arc deposition, sputtering, vapor deposition, ion-beam assisted evaporative deposition or electrospray deposition. With any of these techniques, a sacrificial substrate (e.g., a cylindrical form of copper or a polymer) can be provided for thin-film material deposition and then removed after material deposition.

The thin-film graft 608 can also be made by deposition of a thin film onto a sacrificial two-dimensional substrate (i.e., a planar substrate) then thereafter rolled about a three-dimensional form (i.e., a cylindrical form) and welded together along a common seam to form the preferred configuration (e.g., a hollow thin-film open ended cylinder 600 with perforations). Regardless of the techniques to make prosthetic 600, additional processing may be utilized to enhance the surface finish or physical properties of the thin-film graft. Even though such prosthesis does not have a frame, it is believed that the thin-film material for the graft allows for much greater fatigue life than would be possible using a stent to support the graft. Details of various techniques are shown and described in U.S. Pat. No. 8,460,333, which is incorporated by reference as if set forth herein its entirety in this application.

In one embodiment, bio-active agents can be added to the polymer, the metal alloy of the frame or the thin-film material for delivery to the host's vessel or duct. The bio-active agents may also be used to coat the entire graft, the entire stent or only a portion of either. A coating may include one or more non-genetic therapeutic agents, genetic materials and cells and combinations thereof as well as other polymeric coatings. Non-genetic therapeutic agents include anti-thrombogenic agents such as heparin, heparin derivatives, urokinase, and PPack (dextrophenylalanine proline arginine chloromethylketone); antiproliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet peptides; vascular cell growth promotors such as growth factor inhibitors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms.

Genetic materials include anti-sense DNA and RNA, DNA coding for, anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor alpha and beta, platelet-derived endothelial growth factor, platelet-derived growth factor, tumor necrosis factor alpha, hepatocyte growth factor and insulin like growth factor, cell cycle inhibitors including CD inhibitors, thymidine kinase (“TK”) and other agents useful for interfering with cell proliferation the family of bone morphogenic proteins (“BMPs”), BMP-2, BMP-3, BMP-4, BMP-5, BMP-6 (Vgr-1), BMP-7 (OP-I), BMP-8, BMP-9, BMP-IO, BMP-I, BMP-12, BMP-13, BMP-14, BMP-15, and BMP-16. Desirable BMPs are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA encoding them.

Cells can be of human origin (autologous or allogeneic) or from an animal source (xenogeneic), genetically engineered if desired to deliver proteins of interest at the deployment site. The cells may be provided in a delivery media. The delivery media may be formulated as needed to maintain cell function and viability.

Suitable polymer coating materials include polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof, coatings from polymer dispersions such as polyurethane dispersions (for example, BAYHDROL® fibrin, collagen and derivatives thereof, polysaccharides such as celluloses, starches, dextrans, alginates and derivatives, hyaluronic acid, squalene emulsions. Polyacrylic acid, available as HYDROPLUS® (Boston Scientific Corporation, Natick, Mass.), and described in U.S. Pat. No. 5,091,205, the disclosure of which is hereby incorporated herein by reference, is particularly desirable. Even more desirable is a copolymer of polylactic acid and polycaprolactone. Suitable coverings include nylon, collagen, PTFE and expanded PTFE, polyethylene terephthalate and KEVLAR®, ultra-high molecular weight polyethylene, or any of the materials disclosed in U.S. Pat. No. 5,824,046 and U.S. Pat. No. 5,755,770, which are incorporated by reference herein. More generally, any known graft material may be used including synthetic polymers such as polyethylene, polypropylene, polyurethane, polyglycolic acid, polyesters, polyamides, their mixtures, blends and copolymers.

Referring back to FIGS. 2-4, it is noted that these figures show perforation patterns intended to distribute the restenotic response along a predominant length of the graft 200, 300, or 400. FIG. 2 shows a straight perforation running along a substantial length of the graft 200. By designing a single perforation 208A, it allows for a single region of restenotic response along any cross-section across graft length towards the proximate center of graft 200. Therefore, the remainder of the graft cross-section would be able to maintain patency. FIG. 3 shows a continuous helical perforation 306—this pattern allows a single region of restenotic response per cross-section along graft length towards the proximate center of graft 300. In particular, the perforations of the graft material define a helical path 306, which can be connected from discrete segments 308A, 308B, 308C, 308D and so on from the first graft end to the second graft end. Furthermore, a width of such helical path is progressively smaller as the helical path moves away from the first graft end AO1 or the second graft end AO2 towards the proximate center of the prosthetic 300 so that the open areas AP1, AP2, AP3 and AP4 for each segment is progressively smaller.

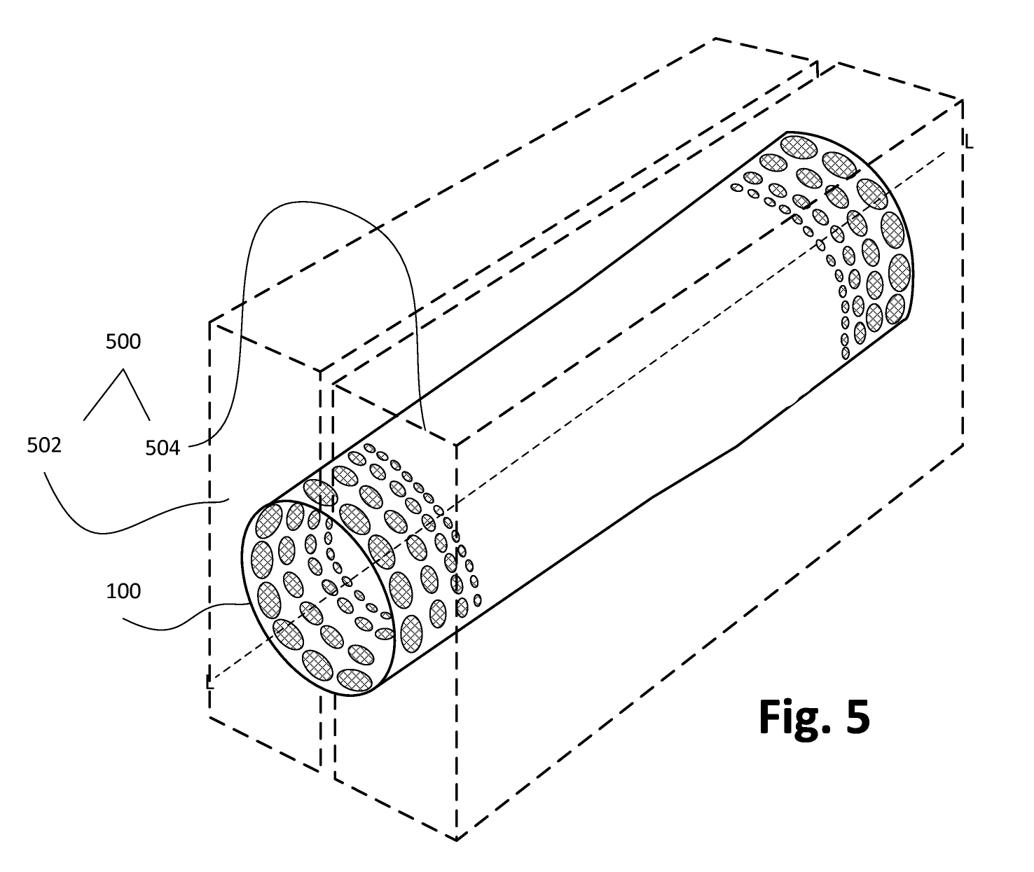

One method of making the endovascular prosthetic embodiments of FIGS. 1-4 can be gleaned from the disclosures relating to FIGS. 9A-9K and 10A-10K of U.S. Pat. No. 6,245,100, which is hereby incorporated by reference herein. It is noted that the perforations can be formed into the graft material with a punch and die with the multiple punches being pre-formed in a split mold form 5-00 shown here in FIG. 5. In FIG. 5, the split form 500 has two halves 502 and 504 and an insert (i.e., a molding form or a rod, not shown) with mating surfaces for the punches formed in the halve 502 and halve 504. The graft can be mounted snugly on the insert rod and the assembly is disposed between the two halves 502 and 504. When the two halves 502 and 504 are clamped together, punches formed on the internal surfaces of each half will punch through the graft material and mate with openings formed on the insert rod. When the split form is removed, perforations are then formed through the graft material such as shown in FIGS. 1-4.

While the invention has been described in terms of particular variations and illustrative figures, those of ordinary skill in the art will recognize that the invention is not limited to the variations or figures described. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well.

What is claimed is:

1. An endovascular prosthetic comprising:
an expandable frame having a plurality of hoops disposed about a longitudinal axis extending through the plurality of hoops from a first frame end to a second frame end;
a generally cylindrical graft material disposed generally coaxial to the expandable frame about the longitudinal axis from a first graft end to a second graft end, the graft material being connected to the frame at a plurality of locations; and wherein the graft material is configured to include perforations formed on the graft material so that the perforations proximate the first and second graft ends are larger than the perforations that are disposed away from the first or second graft end.

2. An endovascular prosthetic comprising:
a generally cylindrical graft material formed via thin-film and disposed generally coaxial to the expandable frame about the longitudinal axis from a first graft end to a second graft end, the material being unsupported by a separate frame; and wherein the graft material is configured to include perforations formed on the graft material so that the perforations proximate the first and second graft ends are larger than the perforations that are disposed away from the first or second graft end.

3. The endovascular prosthetic of claim 1 or claim 2, wherein the perforations proximate the first and second graft ends define respective hoops of perforations proximate the first and second graft ends.

4. The endovascular prosthetic of claim 1 or claim 2, wherein the perforations of the graft material define a helical path from the first graft end to the second graft end and a width of such helical path is progressively smaller as the helical path moves away from the first graft end or the second graft end.

5. The endovascular prosthetic of claim 1, wherein the expandable frame is disposed on an inner surface of the graft material that is facing the longitudinal axis.

6. The endovascular prosthetic of claim 1, wherein the expandable frame is disposed between an inner graft and an outer graft so that the expandable frame is sandwiched between the graft materials.

7. The endovascular prosthetic of claim 1 or claim 2, in which the perforations proximate the first graft end or the second graft end comprise a plurality of perforations wherein each perforation defines an opening having a first aspect ratio from about 0.1 to about 0.5 of an open area of one of the first and second graft end perpendicular to the longitudinal axis.

8. The endovascular prosthetic of claim 5 or claim 2, in which each of the perforations disposed away from one of the first and second graft ends defines an open area having a second aspect ratio that is about 0.4 to about 0.9 times the open area of the perforations proximate one of the first and second graft ends.

9. The endovascular prosthetic of claim 1 or claim 2, in which the perforations comprise at least a slit through the graft material and extending generally parallel to the longitudinal axis.

10. The endovascular prosthetic of claim 9, in which the at least one slit comprises two slits disposed diametrically with respect to the longitudinal axis and spaced apart longitudinally.

11. The endovascular prosthetic of claim 1, in which the expandable frame comprises a self-expanding frame.

12. The endovascular prosthetic of claim 1, in which the expandable frame comprises a balloon expandable frame.

13. The endovascular prosthetic of one of claim 10 or claim 11, in which the frame comprises a bioresorbable material.

14. The endovascular prosthetic of claim 1, in which the frame comprises a series of hoops connected to each other via connectors of the same material as the frame.

15. The endovascular prosthetic of claim 1 in which the frame comprises a series of hoops independent from each other so that the hoops are connected indirectly through the graft material.

16. The endovascular prosthetic of claim 1, in which the graft material comprises a material selected from, PET (polyester), Fluoro-polymers such as PTFE and FEP, spun PTFE, HDPE, and combinations thereof.

17. The endovascular prosthetic of claim 1, in which the first aspect ratio comprises a range from 0.2 to 0.4.

18. The endovascular prosthetic of claim 6, in which the second aspect ratio comprises a range from about 0.5 to about 0.8.

19. The endovascular prosthetic of claim 2, in which the thin-film comprises shape memory materials.

20. The endovascular prosthetic of claim 18, in which the shape memory material comprises nitinol.
